(19) United States
(12) Patent Application Publication
(10) Pub. No.: US 2009/0084984 A1
(43) Pub. Date: Apr. 2, 2009

(54) Irradiation system and method

(75) Inventor: Francisco Miguel Hernandez, Concord, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS USA, INC.

(21) Appl. No.: 12/011,840

(22) Filed: Jan. 29, 2008

Related U.S. Application Data
(60) Provisional application No. 60/995,850, filed on Sep. 28, 2007.

(51) Int. Cl. A61N 5/00

(52) U.S. Cl. 250/492.1

(57) ABSTRACT

The present invention is directed to a system and method for irradiation of an object. A system for irradiating an object may comprise: a) a radiation source,

the radiation source being rotatable about an object to be irradiated and,

a plane defined by the rotation of the radiation source being substantially parallel to a primary axis of the object to be irradiated; b) a processing unit comprising a collimator controller, and c) a collimator having one or more collimator leaves, wherein the collimator controller controls configuration of the one or more collimator leaves, and wherein the configuration of the one or more collimator leaves is determined by a rotational position of the radiation source.

A method for irradiating an object may comprise: a) disposing a radiation source in a first position with respect to an object to be irradiated; b) rotating the radiation source in a first direction about the object to be irradiated, the rotation being in a plane substantially parallel to a primary axis of the object; c) configuring a collimator to block a first portion of the object from the field of view of the radiation source according to a degree of rotation of the radiation source; and d) emitting a first radiation dose.

### BACKGROUND

The present application claims priority to U.S. Patent Application No. 60/995,850, filed on Sep. 28, 2007 and entitled “IRRADIATION SYSTEM AND METHOD”, the contents of which are incorporated herein by reference for all purposes.

Bone marrow transplantation is a medical procedure in the fields of hematology and oncology, most often performed for people with diseases of the blood, bone marrow, or certain types of cancer. Photon radiation therapy may be required as a preconditioning treatment prior to a bone marrow transplant. Such radiation therapy serves to destroy any remaining malignant cells as well as to inhibit the patient's immunologic capabilities to prevent rejection of the transplanted marrow.

Alternately, such radiation therapy may be used for palliative treatments in the case of patients with an extensive number of bone metastasis and, in the case of superficial treatments for mycosis fungoides and Kaposi's sarcoma, the radiation may comprise electron beam radiation.

In order to ensure sufficient coverage of a patient's anatomy, total-body irradiation (TBI) may be utilized. TBI is a process whereby a patient's entire anatomy is subjected to radiation. Common TBI practices generally utilize static radiation source configurations where a patient is required to stand a large distance from the source in order for the field of view to be able to cover his/her complete anatomy. Also, different parts of the body may require specific doses and some parts of the anatomy may require shielding (i.e. lungs).

Due to the low dose rates and static nature of the radiation source and shielding elements utilized for this type of technique, a patient may be required to stand immobile for long periods during set-up and treatment time (on the order of 30 minutes).

Therefore, it would be desirable to provide an improved system and a method for irradiating an object.

### SUMMARY

Accordingly, the present disclosures are directed to a system and method for irradiating an object.

A system for irradiating an object may comprise: a) a radiation source,

the radiation source being rotatable about an object to be irradiated and,

a plane defined by the rotation of the radiation source being substantially parallel to a primary axis of the object to be irradiated; b) a processing unit comprising a collimator controller, and c) a collimator having one or more collimator leaves, wherein the collimator controller controls configuration of the one or more collimator leaves, and wherein the configuration of the one or more collimator leaves is determined by a rotational position of the radiation source.

A method for irradiating an object may comprise: a) disposing a radiation source in a first position with respect to an object to be irradiated; b) rotating the radiation source in a first direction about the object to be irradiated, the rotation being in a plane substantially parallel to a primary axis of the object; c) configuring a collimator to block a first portion of the object from the field of view of the radiation source according to a degree of rotation of the radiation source; and d) emitting a first radiation dose.

It is to be understood that both the foregoing general description and the following detailed description are exemplary and explanatory only and are not restrictive of the invention claimed.

### BRIEF DESCRIPTION OF THE DRAWINGS

The numerous objects and advantages of the present invention may be better understood by those skilled in the art by reference to the accompanying figures in which:

FIG. 1 depicts a system for irradiation of an object;

FIG. 2 depicts a radiation dose rate profile;

FIG. 3A depicts a system for irradiation of an object;

FIG. 3B depicts a system for irradiation of an object;

FIG. 3C depicts a system for irradiation of an object;

FIG. 4A depicts a system for irradiation of an object;

FIG. 4B depicts a system for irradiation of an object;

FIG. 4C depicts a system for irradiation of an object;

FIG. 5A depicts a system for irradiation of an object;

FIG. 5B depicts a system for irradiation of an object;

FIG. 6 depicts process flowchart for a method for irradiation of an object.

### DETAILED DESCRIPTION

The following discussion is presented to enable a person skilled in the art to make and use the present teachings. Various modifications to the illustrated embodiments will be readily apparent to those skilled in the art, and the generic principles herein may be applied to other embodiments and applications without departing from the present teachings. Thus, the present teachings are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present teachings. Skilled artisans will recognize the examples provided herein have many useful alternatives and fall within the scope of the present teachings.

The detailed description provided below is directed to a system and a method of conducting irradiation of an object. Additional details of the invention are provided in the examples illustrated in the accompanying drawings.

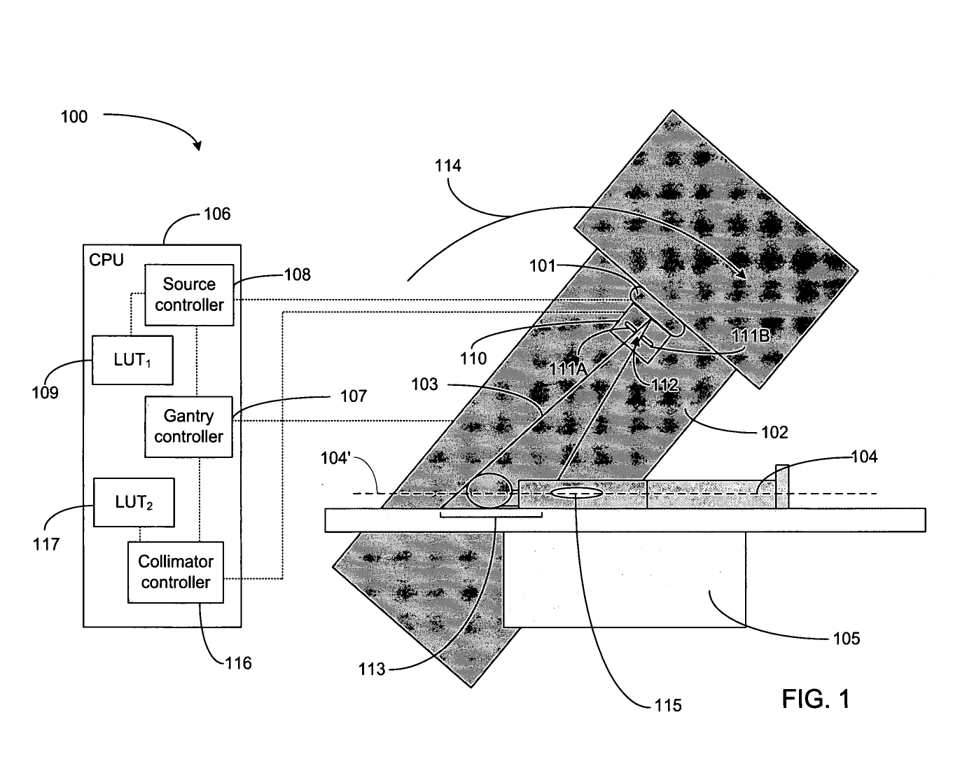

Referring to FIG. 1, a system for total-body irradiation (TBI) 100 is presented. The system 100 may comprise a radiation source 101 affixed to a support structure, such as a gantry 102. The radiation source 101 may generate ionizing radiation 103 (e.g. photon or electron radiation) which may be directed generally towards an object to be irradiated (e.g. a patient 104) disposed on a treatment table 105. Radiation doses for treatment purposes may be on the order of 8-10 Gy which may be applied over multiple fractions. Common dose rates may be on the order of 50 cGy per min and the photon energy may be approximately 6 MV.

The treatment table 105 may be placed a given distance away from the radiation source 101 so as to maximize the radiation field-of-view 113 observed at the location of the patient 104.

It may be the case that the size of a patient's 104 anatomy or the spatial configurations of the room in which the system is to be operated are such that the field-of-view of the radiation 113 which is observed at the location of the patient 104 is insufficient to fully cover the patient 104.

In such cases, the gantry 102 may be configured so as to rotate around a particular axis such that radiation 103 may be provided to the entirety of the patient 104. The patient 104 may be disposed in a position such that the primary axis of the patient's anatomy 104′ is substantially parallel to the plane defined by the rotation 114 of the radiation source 101. With such rotation, it may be necessary to vary the dose rate of radiation 103 during the rotation so as to ensure that appropriate dosages are applied to the entirety of the patient 104.

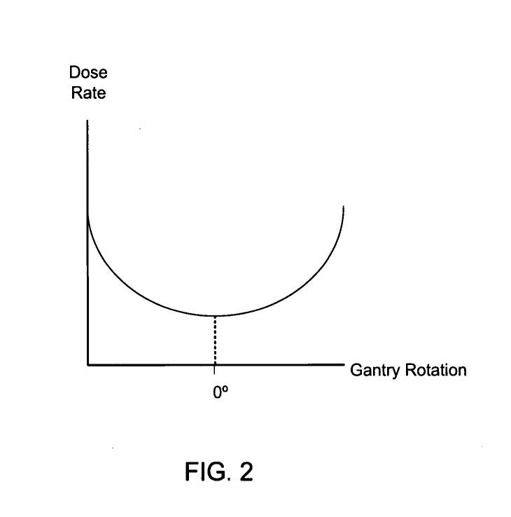

Referring to FIG. 2, a dose rate profile 200 is presented. FIG. 2 depicts a graphical representation of the relationship between the position of the radiation source 101/gantry 102 and the dose rate of radiation 103 being applied. As can be seen, when the gantry 102 is in a position substantially perpendicular to the plane defined by the patient 104 (i.e. 0° rotation), the dose rate may be at its minimum. When the gantry is rotated away from the perpendicular position, the dose rate may be increased. While FIG. 2 is directed to a radiation application scheme predicated on a consistent dose application, one of skill in the art would recognize that the dose rates presented may be varied so as to provide increased or decreased levels of radiation to given portions of the patient 104 anatomy so as to focus on particular treatment localities or to protect sensitive organs.

Referring again to FIG. 1, the system 100 may further comprise a processing unit 106. The processing unit 106 may include a gantry controller 107 and a radiation source controller 108. The gantry controller 107 may comprise application specific integrated circuitry (ASIC), firmware, or software configured run on a microprocessor to control the rotational movement of the gantry 102. The gantry controller 107 may serve to control the speed and/or direction of the rotational movement of the gantry 102 so as to allow for the application of specifically prescribed radiation 103 doses to specific portions of a patient 104.

The radiation source controller 108 may comprise application specific integrated circuitry (ASIC), firmware, or software configured run on a microprocessor to control the firing of the radiation source 101. The radiation source controller 108 may serve to control the dose rate of radiation 103 depending on the position of the gantry 102 (as determined by the gantry controller) so as to allow for the application of varying levels of radiation 103 doses to specific portions of the patient 104.

The system 100 may further comprise a look-up table 109. The look-up table 109 may comprise specific dose rate vs. gantry position data which may be accessed by the radiation source controller 108. The radiation source controller 108 may receive the current position of the gantry 102 from the gantry controller 107 and utilize the look-up table 109 to determine the appropriate dose rate for the given position of the gantry 102. The dose rate vs. gantry position data may be optimized according the physical characteristics (e.g. height, proportions, locations of organs, etc.) of the patient 104. The specific size and orientation of a given patients physical characteristics may result in associated geometric relationships with the various positions of the gantry 102. As such, the dose rate at a given gantry position for a patient 104 having a large physical stature may be different than the dose rate for a patient having a small physical stature. Knowledge of the physical characteristics of a given patient may allow for the optimization of the dose rate vs. gantry position data maintained in the look-up table 109.

During TBI treatments, it may be desirable to prevent irradiation of certain portions of a patient anatomy (e.g. the lungs) due to the sensitivity of those portions or avoid irradiating healthy tissue.

The system 100 may further comprise a collimator 110. The collimator 110 may be a multi-leaf collimator (MLC) including one or more collimator leaves 111. The leaves 111 may be arranged in pairs comprising a first leaf 111A and a second leaf 111 B. The leaves 111 may be adjustable such that they may be positioned in such a manner so as to provide an aperture 112 for the transmission of radiation 103 having a given field-of-view 113 with respect to the location of a patient 104.

During TBI, the gantry 102 may be rotated 114 so as to irradiate certain portions of the patient 104. Portions of the patient 104 which may be desired to be shielded from the radiation 103 (e.g. the lungs 115) may be protected by dynamically shifting one or more of the collimator leaves 111.

While the present description is directed to a collimator 110 having two opposing collimator leaves 111A, 111B, it should be recognized by one skilled in the art that the one-dimensional relationship between leaves 111A and 111B may be easily extended to two-dimensions (e.g. multiple leaves in a common plane) or three-dimensions (e.g. multiple planes of leaves disposed atop one another).

The system 100 may further include a collimator controller 116. The collimator controller may comprise application specific integrated circuitry (ASIC), firmware, or software configured to run on a microprocessor to control the relative positions of the one or more collimator leaves 111. The collimator controller 116 may serve to control the field-of-view 113 of the radiation 103 depending on the position of the gantry 102 (as determined by the gantry controller) so as to allow for the application of radiation 103 doses only to specific portions of the patient 104.

The system 100 may further comprise a look-up table 117. The look-up table may comprise collimator leaf 111 position vs. gantry 102 position data which may be accessed by the collimator controller 116. The collimator controller 116 may receive the current position of the gantry 102 from the gantry controller 107 and utilize the look-up table 117 to determine the appropriate positions of the one or more collimator leaves 111 for a given position of the gantry 102. The collimator leaf 111 position vs. gantry position data may be optimized according the physical characteristics (e.g. height, proportions, locations of organs, etc.) of a patient 104. The specific size and orientation of a given patients physical characteristics may result in associated geometric relationships with the various positions of the gantry 102. As such, the collimator leaf position at a given gantry position for a patient 104 having a large physical stature may be different than the dose rate for a patient having a small physical stature. Knowledge of the physical characteristics of a given patient may allow for the optimization of the collimator position vs. gantry position data maintained in the look-up table 117.

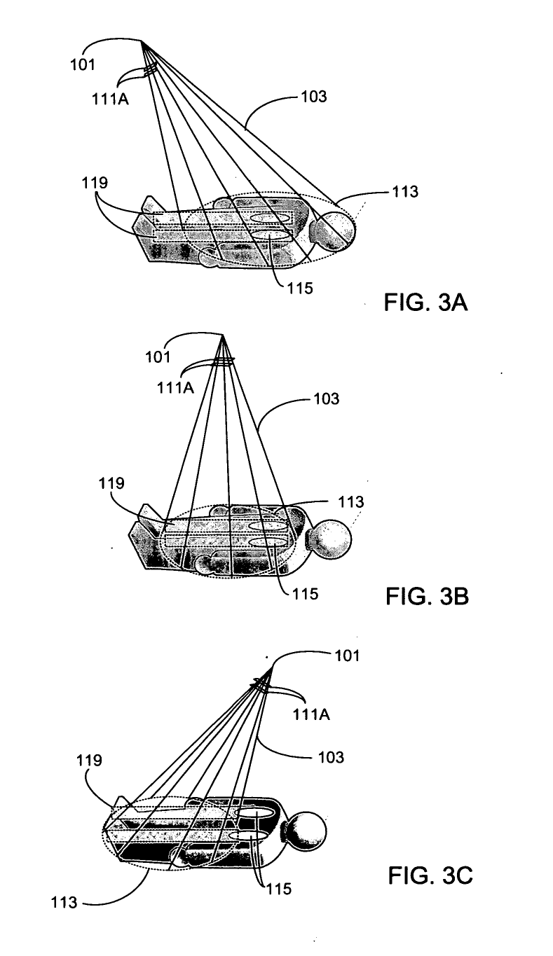

Referring to FIG. 3A, a first position of a radiation source 101, and corresponding positions of a collimator leaves 111A for a first gantry swing are presented. The respective positions of the one or more collimator leaves 111A may result in shielding certain portions 119 of the field-of-view 113 of the radiation source 101 which include portions of a patient anatomy (e.g. the lungs 115) which are not to be irradiated.

During the rotation 114 of the radiation source 101 for a given radiation treatment session, the position of the one or more collimator leaves 111A may be adjusted such that the radiation 103 is continually prevented from encroaching upon portions 119 of the patient to be shielded from the radiation (e.g. the lungs 115).

Referring to FIG. 3B, a second position of the radiation source 101, and corresponding positions of the one or more collimator leaves 111A are presented. During the first gantry swing, the one or more collimator leaves 111A may be moved in a direction substantially opposite that of the rotation of the radiation source. In such a configuration, the one or more collimator leaves 111A shield increasingly larger portions 119 of the field of view 113. Referring to FIG. 3C, further rotation of the radiation source 101 may result in the one or more collimator leaves 11 1A being positioned such that they shield still larger portions 119 of the filed of view 113.

Should further irradiation of a patient be required, additional gantry swings for additional radiation applications may be provided.

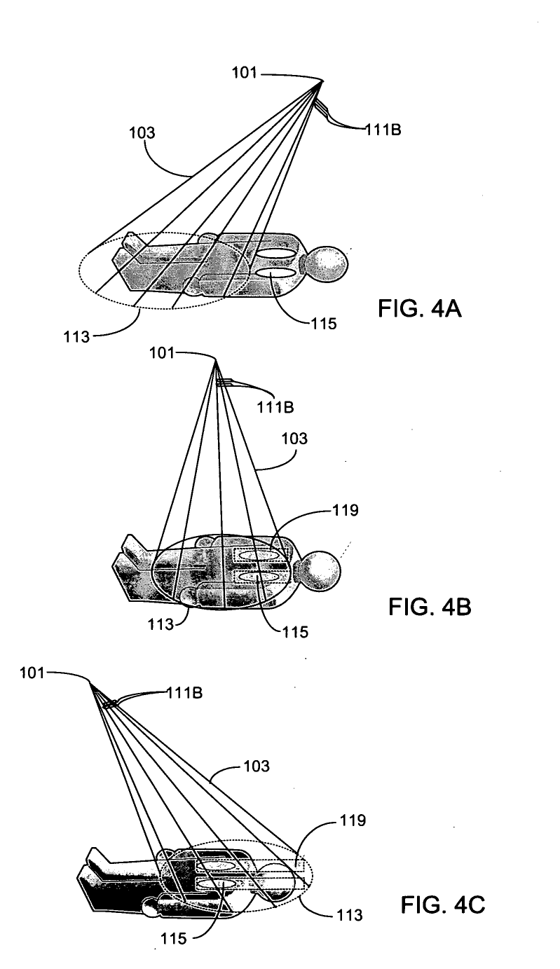

Referring to FIG. 4A, a first position of a radiation source 101, and corresponding positions of second collimator leaves 111B for a second gantry swing are presented. The respective positions of the one or more collimator leaves 111B may result in shielding certain portions 119 of the field-of-view 113 of the radiation source 101 which include portions of a patient anatomy (e.g. the lungs 115) which are not to be irradiated.

Referring to FIG. 4B, a second position of the radiation source 101, and corresponding positions of the one or more collimator leaves 111B are presented. During the second gantry swing, the one or more collimator leaves 111B may be moved in a direction substantially opposite that of the rotation of the radiation source. In such a configuration, the one or more collimator leaves 111 shield increasingly larger portions 119 of the field of view 113. Referring to FIG. 4C, further rotation of the radiation source 101 may result in the one or more collimator leaves 111B being positioned such that they shield still larger portions 119 of the filed of view 113.

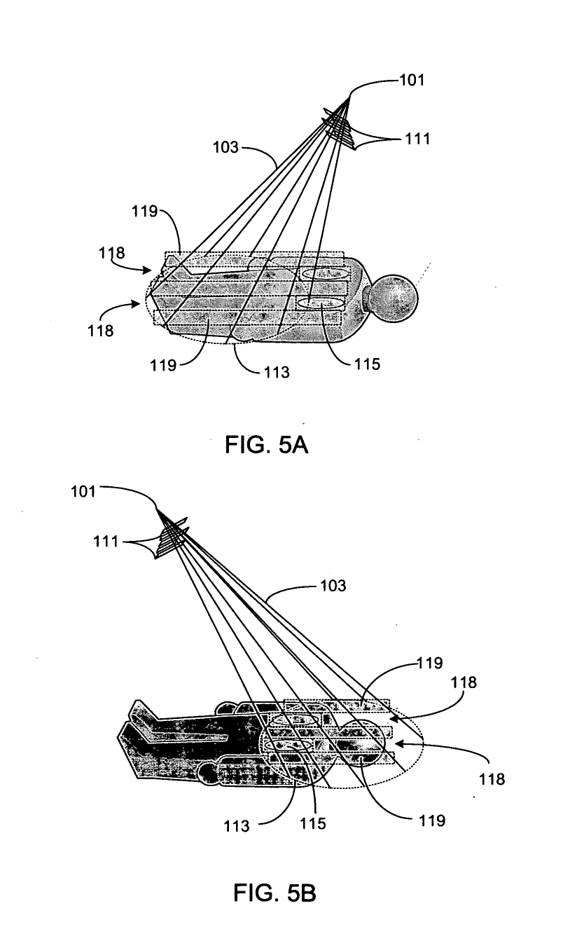

Referring to FIGS. 5A and 5B, various static dosing configurations are presented. In order to irradiate inadequately irradiated portions 118, the radiation source 101 may be rotated into a fixed position such that the radiation 103 is directed towards portions 118. Also, as described above, adjustable collimator leaves 111 may be positioned so as to limit the filed of view 113 of the radiation 103 to the portions 118 and avoid application of additional radiation to portions 119 which have been irradiated during one or more gantry swings or portions 115 which should not be irradiated.

Similarly, the patient 104 may be disposed in a position which is non-orthogonal with respect to the axis of rotation of the gantry 102 (not shown). Likewise, the patient 104 may be placed on their side in a position orthogonal to the axis of rotation of the gantry (not shown). In such alternated patient configurations, additional static and/or dynamic irradiations may be conducted to provide additional coverage of portions 118 which may have previously received inadequate irradiation.

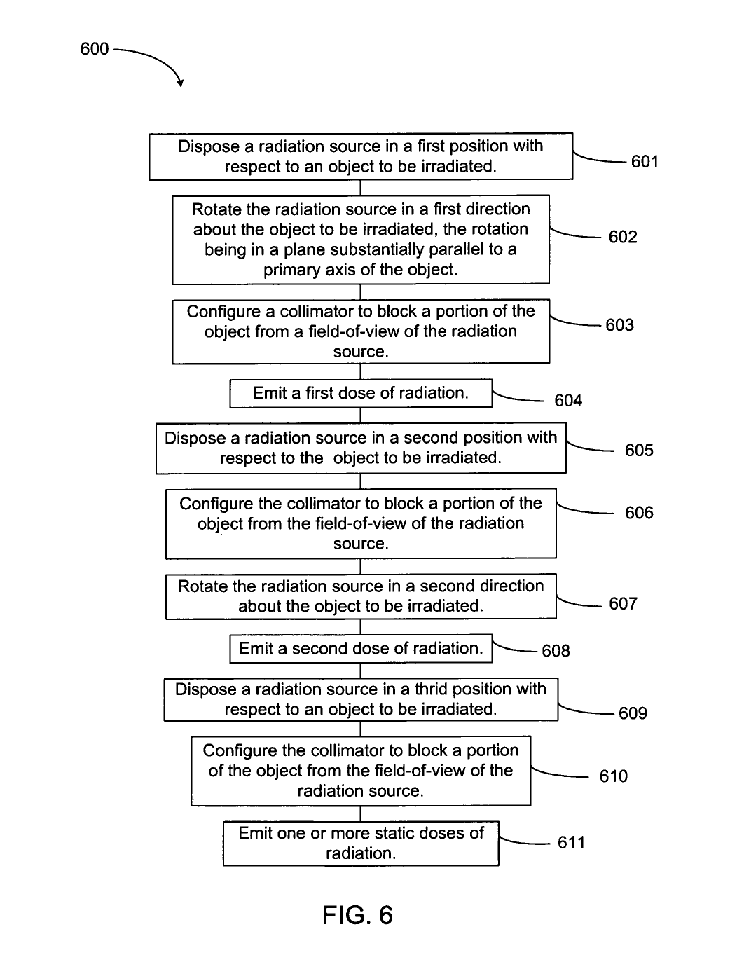

Referring to FIG. 6, a method 600 for TBI is presented. A radiation source may be disposed in a first rotational position with respect to an object (such as a patient) such that the radiation is directed towards the object at step 601.

The radiation source may undergo a first rotation about an axis at step 602. The rotation may be in a plane substantially parallel to a primary axis of the object being irradiated. The rotation of the radiation source may be accompanied by periodic or continuous emission of doses of radiation at step 604. The magnitude of the radiation doses may vary according to the relationship between the rotational position of the radiation source and the position of the object to be irradiated. The radiation dose profile may have an exponential relation to the rotational position of the radiation source with respect to the position of the object to be irradiated.

A collimator may be configured to specify a shield particular region of the object from irradiation during the first rotation of the radiation source at step 603. The collimator may be a multi-leaf collimator (MLC) comprising one or more banks of collimator leaves. The configuration of the collimator may be a dynamic process where the positions of one or more leaves of the MLC are positioned according to the relationship between the rotational position of the radiation source and the position of object being irradiated. During the first rotation of the radiation source, one or more leaves of the MLC may be positioned so as to block a portion of the radiation, thereby protecting certain portions of the object from irradiation. The MLC leaf configurations may be continuously altered according to the degree of rotation of the radiation source to ensure that the radiation field-of-view does not include certain object portions despite the rotation of the radiation source.

Following the first rotation, the radiation source may be disposed in a second rotational position with respect to the object to be irradiated at step 605. A collimator may be configured to shield a particular region of the object from irradiation during a second rotation of the radiation source at step 606. During the second rotation of the radiation source, one or more leaves of the MLC may be positioned so as to block a portion of the radiation, thereby protecting certain portions of the object from irradiation. The MLC leaf configurations may be continuously altered to ensure that the radiation field-of-view does not include certain object portions despite the rotation of the radiation source. The MLCs used during the second rotation of the radiation source may be an opposite bank of MLCs as compared to those used during the first rotation of the radiation source. The radiation source may undergo a second rotation in a direction substantially opposite that of the first rotation at step 607. The rotation of the radiation source may be accompanied by periodic or continuous doses of radiation by the radiation source at step 608.

The radiation source may be disposed in one or more static positions for the delivery of one or more static doses of radiation at step 609. A static position may correspond to an orientation of the radiation source with respect to the object to be irradiated where a portion of the object which was inadequately irradiated during a rotational irradiation (such as those of steps 604 and 608) falls within the field-of-view of the radiation source. One or more static doses of radiation may be emitted at step 611. Similar to steps 602 and 606, during the static radiation dose, one or more leaves of the MLC may be positioned so as to block a portion of the radiation, thereby protecting certain portions of the object from irradiation at step 610.

Similarly, the steps of the method 600 may be implemented as computer readable instructions which may be stored on a computer readable medium. These computer readable instructions may comprise firmware or software and may be executed by a processing device such as an ASIC or a microprocessor so as to accomplish the method steps.

One skilled in the art will recognize that the foregoing components (e.g., steps), devices, and objects in FIGS. 1-6 and the discussion accompanying them are used as examples for the sake of conceptual clarity and that various configuration modifications are common. Consequently, as used herein, the specific exemplars set forth in FIGS. 1-6 and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

While particular embodiments of the subject matter of this application have been shown and described, it will be obvious to those skilled in the art that, based upon the teaching herein, changes and modifications may be made without departing from the subject matter and its broader aspects and, therefore, the appended claims are to encompass within their scope all such changes and modifications as are within the true spirit and scope of the subject matter. Furthermore, it is to be understood that the subject matter of this application is solely defined by the appended claims.

What is claimed is:

1. A system for irradiation of an object, the system comprising:
a radiation source,
the radiation source being rotatable about an object to be irradiated and,
a plane defined by the rotation of the radiation source being substantially parallel to a primary axis of the object to be irradiated;
a processing unit comprising a collimator controller, and
a collimator having one or more collimator leaves,
wherein the collimator controller controls configuration of the one or more collimator leaves, and wherein the configuration of the one or more collimator leaves is determined by a rotational position of the radiation source.

2. The system of claim 1,
wherein rotation of the radiation source irradiates substantially all of the object to be irradiated.

3. The system of claim 1,
the processing unit further comprising:
a radiation source controller.

4. The system of claim 3,
wherein the radiation source controller controls dose rates of radiation generated by the radiation source, and
wherein the dose rates are determined by a rotational position of the radiation source.

5. The system of claim 4,
the processing unit further comprising:
a look-up table, the look-up table comprising a plurality of radiation dose rates corresponding to a plurality of radiation source positions.

6. A method for irradiation of an object, the method comprising the steps:
disposing a radiation source in a first position with respect to an object to be irradiated;
rotating the radiation source in a first direction about the object to be irradiated, the rotation being in a plane substantially parallel to a primary axis of the object;
configuring a collimator to block a first portion of the object from the field of view of the radiation source according to a degree of rotation of the radiation source; and
emitting a first radiation dose.

7. The method of claim 6, further comprising the step:
configuring the collimator to block a second portion of the object from the field of view of the radiation source;

8. The method of claim 6,
wherein the first radiation dose is at a first dose rate;
the method further comprising the step:
emitting a second radiation dose at a second dose rate.

9. The method of claim 6, further comprising the steps:
disposing the radiation source in a second position with respect to the object to be irradiated;
configuring the collimator to block a second portion of the object from the field of view of the radiation source;
rotating the radiation source in a second direction about the object to be irradiated; and
emitting a second radiation dose.

10. The method of claim 6, further comprising the steps:
disposing the radiation source in a second position with respect to the object to be irradiated;
configuring the collimator to block a second portion of the object from the field of view of the radiation source;
emitting a second dose of radiation.

11. The method of claim 6, further comprising the step:
computing a radiation dose rate,
wherein the dose rate is a function of a degree of rotation of the radiation source.

12. A computer-readable storage medium having computer readable instructions for carrying out a method for irradiation of an object, the method comprising the steps:
disposing a radiation source in a first position with respect to an object to be irradiated;
rotating the radiation source in a first direction about the object to be irradiated, the rotation being in a plane substantially parallel to a primary axis of the object;
configuring a collimator to block a first portion of the object from the field of view of the radiation source according to a degree of rotation of the radiation source; and
emitting a first radiation dose.

13. The computer-readable storage medium having computer readable instructions for carrying out a method for irradiation of an object of claim 12, the method further comprising the step:
configuring the collimator to block a second portion of the object from the field of view of the radiation source;

14. The computer-readable storage medium having computer readable instructions for carrying out a method for irradiation of an object of claim 12, wherein the first radiation dose is at a first dose rate;
the method further comprising the step:
emitting a second radiation dose at a second dose rate.

15. The computer-readable storage medium having computer readable instructions for carrying out a method for irradiation of an object of claim 12, the method further comprising the steps:
disposing the radiation source in a second position with respect to the object to be irradiated;
configuring the collimator to block a second portion of the object from the field of view of the radiation source;
rotating the radiation source in a second direction about the object to be irradiated; and
emitting a second radiation dose.

16. The computer-readable storage medium having computer readable instructions for carrying out a method for irradiation of an object of claim 12, the method further comprising the steps:
disposing the radiation source in a second position with respect to the object to be irradiated;
configuring the collimator to block a second portion of the object from the field of view of the radiation source;
emitting a second dose of radiation.

17. The computer-readable storage medium having computer readable instructions for carrying out a method for irradiation of an object of claim 12, the method further comprising the step:
computing a radiation dose rate,
wherein the dose rate is a function of a degree of rotation of the radiation source.
